(19) United States
(12) Patent Application Publication
(10) Pub. No.: US 2004/0015220 A1
(43) Pub. Date: Jan. 22, 2004

(54) Sheet-shaped heating element and method for preparing the same

(76) Inventors: Key-An Um, Kyunggi-do (KR); Sung-Su Hwang, Kyunggi-do (KR); Joon-Gyo Oh, Seoul (KR); Yong-Youn Hwang, Kyunggi-do (KR)

Correspondence Address: Abelman Frayne & Schwab, 150 East 42nd Street, New York, NY, 10017-5612, US

(21) Appl. No.: 10/399,871

(22) PCT Filed: Oct. 23, 2001
(86) PCT No.: PCT/KR01/01819

(30) Foreign Application Priority Data
Oct. 30, 2000 (KR) 2000-63930

(51) Int. Cl. A61F 7/00

(52) U.S. Cl. 607/108

(57) ABSTRACT

Disclosed are a sheet-shaped heating element, which has the advantages of excellent attachability and no lateral displacement of a heat generating composition due to its load, and a method for preparing the same. The sheet-shaped heating element is characterized in that the film or sheet is formed with multiple cells having a height of 0.1-50 mm, a width of 1-100 mm and a distance oxidizable metal powder-containing between cells of 0.1-50 mm, and an oxidizable metal powder-containing heat generating composition is filled in the cells, and a pressure sensitive adhesive or a pharmacologically active material-containing adhesive matrix is attached to the bottom face of the heating element. Such flexible heating element can be attached to bendable portions of humans or animals and is not detached even after the heat generating composition is cured.

### TECHNICAL FIELD

The present invention pertains, in general, to a heating element, which generates heat through a reaction with oxygen in the air, in a gas permeable pouch, and a method for preparing the same. More specifically, the present invention is concerned with a sheet-shaped heating element, capable of being easily attached to the skin, and in particular to bendable portions of the human body or other animals, or to clothing, and a method for preparing the same.

### PRIOR ART

Generally, a heating element is used for warming any portion of the human body by generating heat through a reaction with oxygen in the air, in a gas permeable pouch.

As can be seen in FIG. 1, a conventional heating element comprises a sheet-shaped bag 3 having an air permeable structure to a single side or both sides and a heat generating composition 1 filled in the bag.

However, such heating product is relatively thick and the heat generating composition therein is cured, in other words becomes coagulated and gradually hardened in the process of exothemic reaction. This give rise to unpleasant feel, thereby lowering the sense of comfort and the adhesion force to the skin. Additionally, the heating element is easily detached from the attached portion by movement of the body and thus it is difficult to attach to bendable joint areas. Further, the heating element suffers from the disadvantage in that the hardened heat generating composition is laterally displaced by its load. Namely, when wearing a heating element on the body, the heating composition moves to the bottom of the bag due to its weight, not only during sports but also when standing still. Therefore the heat generation feature becomes less effective.

In order to overcome the above problems, there is provided a heating product made by containing the heat generating composition in the pores of non-woven fabric, embossing such fabric, introducing the embossed fabric into the bag of a structure which allows passage of air through a single side or both sides, and sealing the bag.

Such heating element can be manufactured in a thin form and the heat generating composition is not laterally displaced by its load, but the heating element has the disadvantage of low heating effect due to the small amount of the heat generating composition filled in the element. The heating element is also decreased in flexibility because of tension of the non-woven fabric, and so cannot be attached to bendable joint areas. Also, the heat generating composition is cured as heat is generated, thus the adhesion force to the skin during or after heating is lowered.

Korean Patent Laid-Open No. 99-44351 discloses a heating element product prepared by mixing a heat generating composition with a hot melt adhesive powder, introducing the mixture into the pores of a porous non-woven fabric, superposing another non-woven fabric onto both of the top and bottom faces of the fabric and compressing it under heating.

The above product is advantageous in that the larger amount of heating composition can be contained therein, and that there is no lateral displacement of the heat generating composition, but is disadvantageous because of high preparation costs by using 3 layers of non-woven fabrics and preparation of a thick sheet form. In addition, an additional bag which is attached with an air permeable membrane is required to maintain constant temperature, and the adhesion force is also reduced due to curing of the heat generating composition. The heating element is not flexible and thus cannot be attached to the bendable areas, attributed to curing of the composition.

### DISCLOSURE OF THE INVENTION

Therefore, an object of the present invention is to solve the problems in the prior arts and to provide a sheet-shaped heating element, which has the advantages of attachability to bendable portions of the human body or other animals, or to clothing, no lateral displacement of the heat generating composition due to its load, and excellent adhesion force during or after heat generation.

It is another object of the present invention to provide a method for preparing the heating element.

In accordance with an embodiment of the present invention, there is provided a sheet-shaped heating element comprising a multiple cells-formed film or sheet, wherein each cell is 0.1-50 mm high and 1-100 mm wide and positioned apart from adjacent cells at a distance of 0.1-50 mm, and is filled with an oxidizable metal powder-containing heat generating composition.

In accordance with another embodiment of the present invention, there is provided a method for preparing the sheet-shaped heating element, comprising the following steps of: compressing a film or sheet under heating or vacuum, or by a molder, to form the film or sheet with multiple cells, each cell having a height of 0.1-50 mm, a width of 1-100 mm and a distance between cells of 0.1-50 mm; filling the cells with an oxidizable metal powder-containing heat generating composition, followed by sealing the cell; attaching a pressure sensitive adhesive or a pharmacologically active material-containing adhesive matrix to the bottom face of the heating element.

### BRIEF DESCRIPTION OF THE DRAWING

FIG. 1 shows a cross sectional view of a conventional heating element.

FIG. 2 shows a perspective view of a heating element according to the present invention.

FIG. 3 shows a cross sectional view of a heating element according to the present invention.

FIG. 4 shows a view illustrating a ratio of cell height and distance between cells.

FIG. 5 shows a cross sectional view of a flexible heating element according to the present invention.

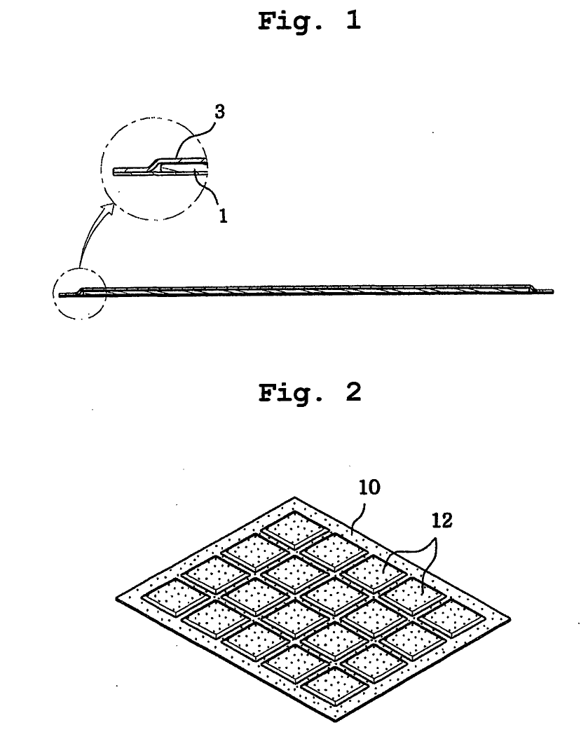

### BEST MODES FOR CARRYING OUT THE INVENTION

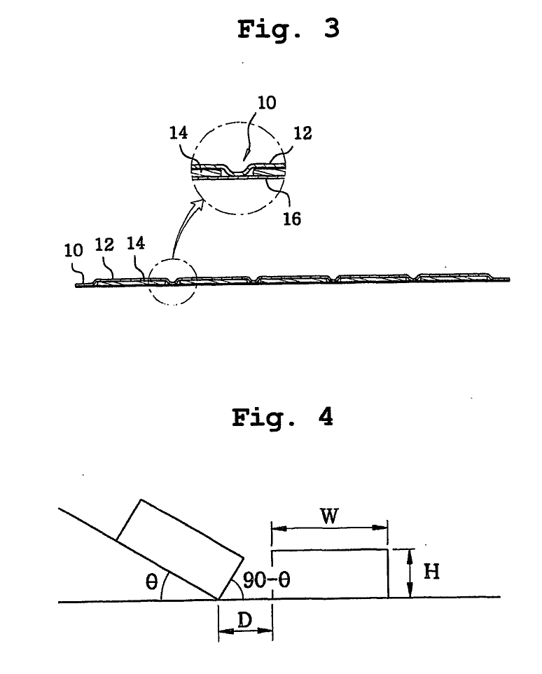

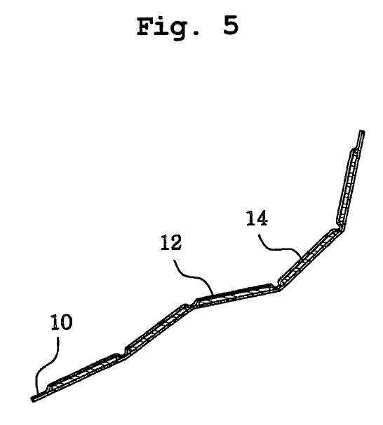

With reference to FIG. 2, there is shown a perspective view of a heating element of the present invention. A cross sectional view of the heating element is shown in FIG. 3, and a height-to-distance ratio of the cell is presented in FIG. 4. As in FIG. 5, a flexible heating element is shown.

Referring to FIGS. 2 and 3, the heating element of the present invention, which is used for warming or heating animals, plants, foods, and further, mechanical equipment, and the human body, can be freely bent and thus be attached to the joints of the human body or bendable portions of machines.

In the present invention, a moisture permeable film or sheet 10 is constructed with a plurality of cells 12, which are made by compressing the moisture permeable film or sheet 10 under heating or vacuum, or by a molder.

The cells 12 are 0.1-50 mm high (H), and 1-100 mm wide (W) and have a distance (D) between cells of 0.1-50 mm, in which the number and height-to-distance ratios of the cell vary with the size of the heating element. Preferably, the cell 12 of 0.5-10 mm height and 3-50 mm width is positioned apart from adjacent cells at a distance of 0.5-30 mm. In addition, the shape of the cell could be a circle, ellipse, or polygon, but is not limited to such specific shapes, and is adjustable for diverse applications.

Also, the distance between cells 12 is not limited, but should be so long as to correspond to 17.4% or more of the height of the cell 12, for attaching to the bendable portions of human bodies. As can be shown in FIG. 4, the cells meet the requirements of height (H)×cos(90−θ)<distance (D), so that they can be freely moved while not coming in contact with adjacent cells. As for the bendable portion having a sloping side of 10°, the distance (D) between cells should be long enough to correspond to 17.4% or more relative to the cell height (H). In the case of the sloping side of 20°, the distance (D) should be long enough to correspond to 34.2% or more relative to the height (H). When the sloping side is inclined to 30°, the distance (D) should be long enough to correspond to 50.0% or more of the height (H). In such a case, the cell does not come in contact with adjacent cells and can be moved freely. Hence, even after the heat generating composition is cured, the flexibility and adhesion force of the heating element can be ensured.

The moisture permeable film or sheet 10 has the thickness varying with end uses. When applied to the foot, the film or sheet is 10-5000 μm thick. The film or sheet is 10-500 μm thick, more specifically, 15-250 μm thick, when directly attached to the body. In general, it is preferred that the thickness of the sheet is 10-2500 μm. The moisture permeable film or sheet 10 has a unit weight of 20-500 g/m², preferably, 30-300 g/m². The moisture permeability is 100-2000 g/m²24 h. The thickness, the unit weight and moisture permeability of the film or sheet 10 are appropriately selected according to purposes, with no limitations.

As the moisture permeable film or sheet 10, use is made of a foamed or defoamed film, sheet, paper, non-woven or woven fabric, or a porous film, sheet or non-woven fabric, in which woven fabric, knitted fabric and non-woven fabric are used as the fabric.

In order to confer air permeability to the defoamed film or sheet made of polymer materials, air vents are formed by elongating the film or sheet, or extracting a specific component from the film or sheet. In addition, air vents can be mechanically formed through drilling or punching micro pins through the film, thus obtaining a porous film or sheet.

As for the foamed film or sheet made of polymer materials, separately formed or serially connected air voids are formed to both faces of the sheet. After the foamed film or sheet is compressed, the air voids within the film or sheet are broken and the top face is communicated with the bottom face, thereby creating air permeability. Also, an air tight film or sheet may be formed.

The cells 12 of thusly formed moisture permeable film or sheet 10 are filled with an oxidizable metal powder-containing heat generating composition 14 and then sealed with non- or permeable film or sheet to be the bottom face.

As a material for the heat generating composition, use can be made of all conventional composition materials, and specifically, oxidizable metal powder, activated charcoal, inorganic electrolytes and water. Concerning the inorganic electrolytes, they are included in the powder composition if mixed as a solid into the heat generating composition above, and if added in the form of aqueous solution after the molding of the sheet, then they are not included into the powder composition.

The oxidizable metal powder is selected from the group consisting of iron, zinc, aluminum, magnesium, or alloys thereof, or mixtures thereof. Of them, iron powder is commonly used, considering safety, handling, price, maintainability and stability. Preferably, iron powder is used, namely reduced iron powder, atomized iron powder, electrolytic powder.

The particle size of the oxidizable metal powder is normally not more than 60 mesh, preferably containing at least 50% of particles which are not larger than 100 mesh.

The blending ratio for the whole of the heat generating composition cannot be specified in general, as it depends upon the properties and shape of the film or sheet to be used and the heat generating function to be achieved. As an example, the heat generating composition 14 comprises 5-30 parts by weight of activated charcoal, 1.5-10 parts by weight of inorganic electrolyte and 25-70 parts by weight of water on the basis of 100 parts by weight of the oxidizable metal powder.

In addition, it is also possible to mix a water retention agent, such as pearlite powder, vermiculite or higher aqueous resin, hydrogen generation depressor or anti-caking agent, as desired.

Onto the bottom face of the sheet-shaped heating element, a pressure sensitive adhesive or a pharmacologically active material-containing adhesive matrix is attached.

The adhesive layer may be in the form of a solvent, emulsion, or hot-melt.

The adhesive layer is made of a material selected from the group consisting of rubber, vinyl acetate, ethylene-vinyl acetate, polyvinyl alcohol, polyvinyl acetal, vinyl chloride, acryl, polyamide, polyethylene, cellulose, polysulfide or hot-melt polymer. In particular, taking into account proper skin-attachability, low skin irritation, and high adhesion force under heat generation, it is preferred that the adhesive layer is formed by rubber, acryl or hot-melt polymer.

The adhesive layer contains cataplasm and thus is useful applicable for a wet dressing layer, or contains transdermal absorption drug and plays a part in a drug-containing layer, whereby a wet dressing effect or therapeutic or pharmacological effect by drug can be obtained under heating.

In the heating element of the present invention, blending of a transdermal absorption drug into the adhesive layer is essential to improve the topical or whole therapeutic effect and to increase the administration effect, because drug delivery to the body is improved by increased blood circulation under heating.

Examples of the transdermal absorption drug include, but are not limited to, epispastics, anti-inflammatory agents, central nervous agents (e.g., hypnotics, antiepileptics, psycholeptics), diuretics, hypotensive drugs, vasodilatins, antitussives, antihistamines, drugs for dysrhythmia, cardiotonics, adrenal cortical hormone agents, topical anaesthetics. These drugs are used alone or in combinations thereof.

Additionally, the present invention provides a method for preparing a sheet-shaped heating element having multiple cells.

The moisture permeable film or sheet 10 is compressed under heating or vacuum, or by using a molder, to form multiple cells 12, which are 0.1-50 mm high, 1-100 mm wide and 0.1-50 mm apart. Preferably, each cell 12 has a height of 0.5-10 mm, a width of 3-50 mm and the distance between cells of 0.5-30 mm. The cell size depends on various applications, without specific limitations.

The cell 12 is filled with an oxidizable metal powder-containing heat generating composition and then sealed with non- or permeable film or sheet to be the bottom face.

Thereafter, a pressure sensitive adhesive 16 or a pharmacologically active material-containing adhesive matrix is attached to the bottom face of the sheet.

During the above procedure, water or an aqueous inorganic electrolyte solution may be selectively added to the heat generating powder composition. More specifically, water or an aqueous inorganic electrolyte solution may be mixed together with the other components before filling the multiple cells fomed in a sheet or film, or added to the powder composition through a spray or an injector immediately before sealing of the cell filled with the other heat generating composition or before vacuum-packaging of the prepared sheet-shaped heating element 10.

Thereby, as can be seen in FIG. 5, even though the heat generating composition 14 becomes hardened during heat generation, the cell compartments 12 can be freely flexed and adhesion force to the skin can be maintained. After a conventional heating element is attached to a bendable portion of the body, the heat generating composition is cured over time and thus detached from the attached portion. Whereas, in the present heating element, even though the heat generating composition filled in the cells 12 is hardened, the distance between cells is so long that the cells can be freely moved while not coming in contact with adjacent cells, thereby maintaining the adhesion force.

In addition, since the heat generating composition 14 is in the self-contained cell, it is not laterally displaced due to the load after the heating element is attached to the body.

A better understanding of the present invention may be obtained in light of the following examples which are set forth to illustrate, but are not to be construed to limit the present invention.

### PREPARATION EXAMPLE 1

47 wt % of iron powder, 10 wt % of activated charcoal, 8 wt % of vermiculite, 5 wt % of wood flour and 2 wt % of sodium chloride were mixed and added with 28 wt % of water, to prepare a heat generating composition, which generates heat through a reaction with oxygen in a air, in a gas permeable pouch.

### EXAMPLES 1-11

In the example 1, a moisture permeable sheet (supplied from Korea Vilene Co., Ltd., moisture permeability 450±50 g/m²24 h) was compressed using a molder, to form 14 square cells of a height of 1 mm, a width of 20 mm and a distance between cells of 3 mm. Then, each of the cells was filled with the heat generating composition obtained from the above preparation example 1 and sealed with non- or permeable film or sheet to be the bottom face, thereby preparing a heating element with multiple-cells. A pressure sensitive adhesive acrylate solution (Monsanto Co., Gelva 737) was applied onto a release liner at a thickness of 300 μm and dried at 70° C. for 10 minutes, to make an adhesive matrix, which was then trasfered onto the bottom face of the above heating element which was cut to suitable sizes, to prepare a heating element capable of being attached onto humans or animals.

The preparation of Examples 2 to 11 were carried out in the same manner as in the example 1, except that height, width and distance between cells varied according to the following table 1, to prepare moisture permeable sheets having multiple cells.

TABLE 1

Cell Size

| | Height | Width | Distance | Cell |
|---|---|---|---|---|
| Ex. No. | (H, mm) | (W, mm) | (D, mm) | Numbers |
| 1 | 1 | 20 | 2 | 20 |
| 2 | 3 | 20 | 2 | 20 |
| 3 | 5 | 20 | 2 | 20 |
| 4 | 10 | 20 | 2 | 20 |
| 5 | 3 | 5 | 3 | 140 |
| 6 | 3 | 10 | 3 | 56 |
| 7 | 3 | 30 | 3 | 9 |
| 8 | 3 | 50 | 3 | 4 |
| 9 | 3 | 20 | 1 | 20 |
| 10 | 3 | 20 | 10 | 12 |
| 11 | 3 | 20 | 20 | 8 |

### EXAMPLE 12

A moisture permeable sheet (supplied from Korea Vilene Co., Ltd., moisture permeability 450±50 g/m²24 h) was compressed by use of a molder, to form 20 square cells having a height of 2 mm, a width of 20 mm and a distance between cells of 1.8 mm. Then, each of the cells was filled with the heat generating composition obtained in the preparation example 1 and then sealed with non- or permeable film or sheet to be the bottom face, thereby preparing a heating element with multiple cells. A pressure sensitive adhesive polyisobutylene solution (National Starch Co., Duro-Tak 87-6430) was applied onto a release liner at a thickness of 300 μm and dried at 70° C. for 10 minutes, to make an adhesive matrix, which was then trasfered onto the bottom side of the above heating element which was cut to suitable sizes, to prepare a heating element capable of being attached onto humans or animals.

### EXAMPLE 13

A moisture permeable sheet (supplied from Korea Vilene Co., Ltd., moisture permeability 450±50 g/m²24 h) was compressed by a molder, to form 30 square cells being 1.2 mm high, 15 mm wide and 2.0 mm apart. Then, each of the cells was filled with the heat generating composition obtained in the preparation example 1 and sealed with non- or permeable film or sheet to be the bottom face, thereby preparing a heating element with multiple cells. 0.4 g of piroxicam was dissolved in 1.8 g of dimethylsulfoxide and 1.5 g of diethyleneglycol monoethylether, and added with 0.3 g of laurdiethanol amide and 0.5 g of polyethyleneglycol 200 monolaurate and mixed well. Thusly obtained solution was mixed with 11 g of a pressure sensitive adhesive polyisobutylene solution (National Starch Co., Duro-Tak 87-2852), left to stand and defoamed. Such solution was applied onto a release liner at a thickness of 400 μm and dried at 70° C. for 10 minutes, to make a pharmacologically active material-containing adhesive matrix, which was then trasfered onto the bottom side of the above heating element which was cut to suitable sizes, to prepare a heating element capable of being attached onto humans or animals.

### EXAMPLE 14

A moisture permeable sheet (supplied from Korea Vilene Co., Ltd., moisture permeability 450±50 g/m²24 h) was compressed using a molder, to form 20 circular cells of a height of 1.0 mm, a width (diameter) of 20 mm and a distance of 1.5 mm. Then, each of the cells was filled with the heat generating composition obtained in the preparation example 1 and sealed with non- or permeable film or sheet to be the bottom face, thereby preparing a heating element with multiple cells. 0.8 g of ketoprofen was dissolved in 1.0 g of ethylalcohol and added with 1.0 g of propylene glycol and 1.0 g of propylene glycol monolaurate and mixed well. Thusly obtained solution was mixed with 12 g of a pressure sensitive adhesive acrylate solution (Monsanto Co., Gelva 1430), left to stand and defoamed. Such solution was applied onto a release liner at a thickness of 300 μm and dried at 70° C. for 10 minutes, to make a pharmacologically active material-containing adhesive matrix, which was then trasfered onto the bottom side of the above heating element which was cut to suitable sizes, to prepare a heating element capable of being attached to human beings or animals.

### EXAMPLE 15

A moisture permeable sheet (supplied from Korea Vilene Co., Ltd., moisture permeability 450±50 g/m²24 h) was compressed by a molder, to form 30 rectangular cells having a height of 1.5 mm, a width of 18 mm, a length of 14 mm and a distance between cells of 2 mm. Then, each of the cells was filled with the heat generating composition obtained in the preparation example 1 and sealed with non- or permeable film or sheet to be the bottom face, thereby preparing a heating element with multiple cells. 0.5 g of ketorolac was dissolved in 2.0 g of ethyl alcohol and added with 0.8 g of propylene glycol and 1.0 g of propylene glycol monolaurate and mixed well. Thusly obtained solution was mixed with 12 g of a pressure sensitive adhesive acrylate solution (Monsanto Co., Gelva 737+Gelva 788 (mixture ratio 1:1)), left to stand and defoamed. Such solution was applied onto a release liner at a thickness of 350 μm and dried at 70° C. for 10 minutes, to make a pharmacologically active material-containing adhesive matrix, which was then trasfered onto the bottom side of the above heating element and cut to suitable sizes, to prepare a heating element capable of attaching to human beings or animals.

### EXAMPLE 16

A moisture permeable sheet (supplied from Korea Vilene Co., Ltd., moisture permeability 450±50 g/m²24 h) was compressed by a molder, to form 20 elliptical cells being 1.7 mm high, 24 mm long wide, 17 mm short wide and 1.3 mm apart. Then, each of the cells was filled with the heat generating composition obtained in the preparation example 1 and sealed with non- or permeable film or sheet to be the bottom face, thereby preparing a heating element with multiple cells. 0.5 g of ibuprofen was dissolved in 1.0 g of ethyl alcohol and N-methylpyrrolidone and mixed with 11 g of a pressure sensitive adhesive acrylate solution (Monsanto Co., Gelva 737), left to stand and defoamed. Such solution was applied onto a release liner at a thickness of 320 μm and dried at 70° C. for 10 minutes, to make a pharmacologically active material-containing adhesive matrix, which was then trasfered onto the bottom side of the above heating element which was cut to suitable sizes, to prepare a heating element capable of being attached to humans or animals.

### COMPARATIVE EXAMPLE 1

The heating elements having multiple cells prepared as in the above examples 1 to 16, and commercially available heating elements were attached to the wrist area (forearm) as a large bendable joint area, and to a flat area (back). Thereafter, the heating elements were examined for attachability during heating, flexibility after heating, lateral displacement of the heat generating composition by its load, and comfort of users. The results are given in Table 2, below.

TABLE 2

| | Attacha- | | Flexi- | | Displace- | | | |
|---|---|---|---|---|---|---|---|---|
| | bility¹⁾ | | bility²⁾ | | ment³⁾ | | Comfort⁴⁾ | |
| | Fore- | | Fore- | | Fore- | | Fore- | |
| Test material | arm | Back | arm | Back | arm | Back | arm | Back |
| 1 | ⊚ | ⊚ | ⊚ | ⊚ | ⊚ | ⊚ | ⊚ | ⊚ |
| 2 | ⊚ | ⊚ | ⊚ | ⊚ | ⊚ | ⊚ | ⊚ | ⊚ |
| 3 | ⊚ | ⊚ | ⊚ | ⊚ | ⊚ | ⊚ | ⊚ | ⊚ |
| 4 | ⊚ | ⊚ | ◯ | ◯ | ◯ | ◯ | Δ | ⊚ |
| 5 | ⊚ | ⊚ | ⊚ | ⊚ | ⊚ | ⊚ | ⊚ | ⊚ |
| 6 | ⊚ | ⊚ | ⊚ | ⊚ | ⊚ | ⊚ | ⊚ | ⊚ |
| 7 | ◯ | ⊚ | ⊚ | ⊚ | ⊚ | ⊚ | ◯ | ⊚ |
| 8 | ◯ | ◯ | ⊚ | ⊚ | ◯ | ◯ | Δ | ◯ |
| 9 | ◯ | ⊚ | ◯ | ◯ | ⊚ | ⊚ | Δ | ⊚ |
| 10 | ⊚ | ⊚ | ⊚ | ⊚ | ⊚ | ⊚ | ⊚ | ⊚ |
| 11 | ⊚ | ⊚ | ⊚ | ⊚ | ⊚ | ⊚ | ⊚ | ⊚ |
| 12 | ⊚ | ⊚ | ⊚ | ⊚ | ⊚ | ⊚ | ⊚ | ⊚ |
| 13 | ⊚ | ⊚ | ⊚ | ⊚ | ⊚ | ⊚ | ⊚ | ⊚ |
| 14 | ⊚ | ⊚ | ⊚ | ⊚ | ⊚ | ⊚ | ⊚ | ⊚ |
| 15 | ⊚ | ⊚ | ⊚ | ⊚ | ⊚ | ⊚ | ⊚ | ⊚ |
| 16 | ⊚ | ⊚ | ⊚ | ⊚ | ⊚ | ⊚ | ⊚ | ⊚ |
| Jikabari | Δ | ◯ | X | X | Δ | Δ | Δ | ⊚ |
| (Hishamitsu | | | | | | | | |
| K.K., Japan) | | | | | | | | |
| Onpas (Mycoal | X | Δ | ◯ | ◯ | X | X | X | ⊚ |
| K.K., Japan) | | | | | | | | |
| Hottosapoto | X | Δ | X | X | ⊚ | ⊚ | X | ◯ |
| (Pionic K.K., | | | | | | | | |
| Japan) | | | | | | | | |

Note:
¹⁾Attachability
⊚: good attachability
◯: detachment from less than 10% of heating element area
Δ: detachment from 10-30% of heating element area
X: detachment from exceeding 30% of heating element area
²⁾Flexibility
⊚: heating element flexible at 60° or more
◯: heating element flexible at 30-60°
Δ: heating element flexible at less than 30°, but cured heat generating composition is broken
Δ: when heating element is flexed, cured heat generating composition is broken
³⁾Displacement
⊚: original shape of heat generating composition is maintained
Δ: parts of heat generating composition are laterally displaced by its load
X: most heat generating composition is laterally displaced by its load
⁴⁾Comfort
⊚: good sense of comfort, natural motion
◯: slightly discomfort, somewhat unnatural motion
Δ: considerable discomfort, some parts are detached when moved
X: extreme discomfort, detachment of 30% or more when moved.

### INDUSTRIAL APPLICABILITY

The heating element of the present invention is freely flexible and thus can be attached to bendable portions of humans or animals. Even though the heat generating composition is hardened after attachment, the heating element is not detached and is advantageous in terms of attachability and no lateral displacement of the heat generating composition.

The present invention has been described in an illustrative manner, and it is to be understood that the terminology used is intended to be in the nature of description rather than of limitation. Many modifications and variations of the present invention are possible in light of the above teachings. Therefore, it is to be understood that within the scope of the appended claims, the invention may be practiced otherwise than as specifically described.

1. A sheet-shaped heating element, comprising a multiple cell-formed film or sheet, wherein each cell is 0.1-50 mm high and 1-100 mm wide and positioned apart from adjacent cells at a distance of 0.1-50 mm, and is filled with an oxidizable metal powder-containing heat generating composition.

2. The heating element as defined in claim 1, wherein the distance between cells is long enough to correspond to 17.4% or more of the cell height.

3. The heating element as defined in claim 1, wherein, as the film or sheet, use is made of a single layer or multiple layers of a foamed or defoamed film, sheet, paper, non-woven, woven fabric or knitted fabric.

4. The heating element as defined in claim 1, wherein a pressure sensitive adhesive or a pharmacologically active material-containing adhesive matrix is attached onto the bottom face of the heating element.

5. A method for preparing the sheet-shaped heating element, comprising the following steps of:
compressing a film or sheet under heating or vacuum, or by a molder, to form the film or sheet with multiple cells, each cell having a height of 0.1-50 mm, a width of 1-100 mm and a distance between cells of 0.1-50 mm;
filling an oxidizable metal powder-containing heat generating composition in the cell, followed by sealing the cell; and
attaching a pressure sensitive adhesive or a pharmacologically active material-containing adhesive matrix onto the bottom face of the heating element.
